(19) United States
(12) Patent Application Publication
(10) Pub. No.: US 2023/0282370 A1
(43) Pub. Date: Sep. 7, 2023

(54) TELEHEALTH PLATFORM WITH ENHANCED MULTI-LEVEL CONSULTATION MATCHING AND COMMAND CENTER

(71) Applicant: TELADOC HEALTH, INC., Purchase, NY (US)

(72) Inventors: Keith Weinberg, Chicago, IL (US); Peter H. Burkhardt, Fort Collins, CO (US); Ari Rasekh, New Rochelle, NY (US)

(21) Appl. No.: 17/994,160

(22) Filed: Nov. 25, 2022

Related U.S. Application Data
(60) Provisional application No. 63/264,553, filed on Nov. 24, 2021.

(51) Int. Cl. G16H 80/00; G16H 40/20

(52) U.S. Cl. CPC G16H 80/00; G16H 40/20

(57) ABSTRACT

To address the complexity associated with matching telehealth consult requests with appropriate providers, a telehealth platform in accordance with the disclosure employs a multi-level matching system that first divides a set of consultation requests into one or more subsets or “virtual waiting rooms” having certain consultation criteria in common. The matching system then orders or prioritizes the requests within each virtual waiting room according to certain other criteria, such as urgency. Providers are assigned to one or more waiting rooms based on whether their provider profiles satisfy the consultation criteria of each waiting room. In addition, the set of providers assigned to a virtual waiting room may be ordered or prioritized according to further criteria. The highest priority provider assigned to a virtual waiting room may then be matched with the highest priority consultation request within that waiting room.

### CROSS-REFERENCE TO RELATED APPLICATIONS

This application claims the benefit of U.S. Provisional Application No. 63/264,553, filed Nov. 24, 2021, which is hereby incorporated by reference in its entirety.

### BACKGROUND

Healthcare providers and patients increasingly need or prefer to consult each other virtually, using communication devices, in lieu of in-person visits—a practice commonly referred to as telemedicine or telehealth. A central problem in telehealth is connecting the right parties at the right time.

A patient often needs to connect with a provider having both a particular specialty and a license to practice in the patient's jurisdiction (e.g., a state). Beyond these requirements, the patient may need to connect with a provider associated with a certain medical group or payment provider. The patient may also prefer to connect with a provider who speaks a particular language or is of a particular demographic background. Numerous other requirements and/or preferences may limit the pool of providers who can service the patient's request. Meanwhile, a provider may have more than one specialty and be licensed in multiple jurisdictions, further complicating the question of which provider should service a request for a particular specialty in a particular jurisdiction. Moreover, although telehealth consults are often between a patient and a healthcare provider, telehealth consults can also be between healthcare providers. For example, an attending physician treating a possible stroke patient in an emergency department may request a consultation with a neurologist located elsewhere.

As to timing, while scheduled telehealth visits are common, patients generally expect to be connected with a provider on-demand, within minutes—not hours—of requesting a consult. In general, as the demand for telehealth grows in each dimension (e.g., frequency, geography, specialty, urgency, etc.), so does the complexity of connecting the right parties at the right time.

### SUMMARY

These and other challenges associated with telehealth delivery are addressed by the following disclosure, an aspect of which is an improved telehealth platform that more efficiently connects people requesting consultations with providers who can service those requests.

To address the complexity associated with matching telehealth consult requests with appropriate providers, a telehealth platform in accordance with the disclosure employs a multi-level matching system that first divides a set of consultation requests into one or more subsets or “virtual waiting rooms” having certain consultation criteria in common. The matching system then orders or prioritizes the requests within each virtual waiting room according to certain other criteria, such as urgency. Providers are assigned to one or more waiting rooms based on whether their provider profiles satisfy the consultation criteria of each waiting room. In addition, the set of providers assigned to a virtual waiting room may be ordered or prioritized according to further criteria. The highest priority provider assigned to a virtual waiting room may then be matched with the highest priority consultation request within that waiting room.

The various criteria used to create virtual waiting rooms, order the requests within a virtual waiting room, and order the providers that can service a virtual waiting room may be configurable. For example, the prioritization scheme used to order requests within one or more waiting rooms may be changed depending on the season (e.g., flu season, slow season, etc.), public health condition (e.g., pandemic), or other conditions.

Application software residing on a server in communication with the matching system may then initiate a communication session between devices associated with the matched provider and consultation request. The matching system may distinguish between providers who are merely logged into the system and those who are actually available to service consult requests. For example, the system may define an available provider as one who is currently servicing requests or has accepted a consultation request within a predetermined period of time. Alternatively or additionally, a provider who has missed or ignored a predetermined number of consultation requests may be designated as unavailable, despite being logged in to the system. By way of example, when a provider is matched with a consultation request, he or she may have a predetermined period of time to accept the request (e.g., 90 seconds). If the provider does not accept the request within that period of time, the request is returned to the virtual waiting room for assignment to another provider. If this sequence occurs, e.g., three consecutive times for a particular provider, the matching system may change the provider's status to unavailable and cease matching that provider with consultation requests until the provider logs in again or manually changes their status. This can reduce overall wait times by limiting the number of times consultations can be matched with providers who may have stepped away from their device.

The matching system may be in communication with a command center. The command center may receive real-time or near real-time information from the matching system. The command center may analyze information from the matching system to identify potential shortages in available providers as well as shortages in available consults for available providers. The command center may include a user interface that displays various metrics based on the information from the matching system and alerts and/or notifications to command center staff relating to potential problems. In one embodiment, when the command center detects a current or impending shortage of online providers assigned to one or more waiting rooms, the command center may retrieve a list of providers that can potentially service the requests in that waiting room and automatically invite those providers to log in and begin servicing those requests. Additionally or alternatively, the command center may display the list so that command center personnel may reach out to the providers manually.

The command center may also be configured to allow administrators to change, configure, or otherwise manage routing rules or other parameters that control which providers are given priority under various conditions. In general, the command center allows command center personnel to anticipate supply and demand issues and work proactively to alleviate or mitigate those issues. In addition, the command center may be configured to automatically take certain actions to alleviate or mitigate such issues. Finally, the command center interfaces with the matching system and enables command center personnel to manually or automatically reconfigure various aspects of the matching system, including waiting room criteria, routing rules, provider prioritization, etc.

Thus, one aspect of the disclosure is a method for establishing electronic communication sessions between patients and healthcare providers. The method comprises receiving, at a server via a network, a plurality of consultation requests from a plurality of patient devices, each request including patient-supplied consultation criteria; identifying, using a processor, each unique combination of consultation criteria among the plurality of consultation requests received at the server; generating, within the server, a virtual waiting room for each unique combination of consultation criteria; assigning, within the server, each consultation request of the plurality of consultation requests to the virtual waiting room with matching consultation criteria; comparing, using the processor, the unique combination of consultation criteria of each virtual waiting room with a profile for each provider stored in a provider database, each provider having a respective provider device; assigning each provider whose profile satisfies a unique combination of consultation criteria to a corresponding virtual waiting room within the server; sorting, using the processor, the consultation requests for each virtual waiting room according to a consultation priority scheme; and establishing, via the network, an electronic communication session between a patient device associated having a highest priority consultation request within each virtual waiting room and a provider device associated with a first available provider assigned to the corresponding virtual waiting room.

Another aspect of the disclosure is a system for establishing electronic communication sessions between patients and healthcare providers. The system comprises a matching server in communication with a plurality of patient devices and a plurality of provider devices. The matching server is configured to perform the following: receive, via a network, a plurality of consultation requests from a plurality of patient devices, each request including patient-supplied consultation criteria; identify, using a processor, each unique combination of consultation criteria among the plurality of consultation requests; generate a virtual waiting room for each unique combination of consultation criteria; assign each consultation request of the plurality of consultation requests to the virtual waiting room with matching consultation criteria; compare, using the processor, the unique combination of consultation criteria of each virtual waiting room with a profile for each provider stored in a provider database, each provider having a respective provider device; assign each provider whose profile satisfies a unique combination of consultation criteria to a corresponding virtual waiting room; sort, using the processor, the consultation requests for each virtual waiting room according to a consultation priority scheme; and establish, via the network, an electronic communication session between a patient device associated having a highest priority consultation request within each virtual waiting room and a provider device associated with a first available provider assigned to the corresponding virtual waiting room.

Yet another aspect of the disclosure is a system for establishing electronic communication sessions between patients and healthcare providers. The system comprises a management terminal in communication with a matching server in communication with a plurality of patient devices and a plurality of provider devices. The matching server is configured to perform the following: receive, via a network, a plurality of consultation requests from a plurality of patient devices, each request including patient-supplied consultation criteria; identify, using a processor, each unique combination of consultation criteria among the plurality of consultation requests; generate a virtual waiting room for each unique combination of consultation criteria; assign each consultation request of the plurality of consultation requests to the virtual waiting room with matching consultation criteria; compare, using the processor, the unique combination of consultation criteria of each virtual waiting room with a profile for each provider stored in a provider database, each provider having a respective provider device; assign each provider whose profile satisfies a unique combination of consultation criteria to a corresponding virtual waiting room; sort, using the processor, the consultation requests for each virtual waiting room according to a consultation priority scheme; and establish, via the network, an electronic communication session between a patient device associated having a highest priority consultation request within each virtual waiting room and a provider device associated with a first available provider assigned to the corresponding virtual waiting room; wherein the management terminal is configured to display a graphical user interface that provides an alert when a condition is met.

### BRIEF DESCRIPTION OF THE DRAWINGS

FIG. 1 is a diagram of a telehealth platform in accordance with the disclosure.

FIG. 2 is a diagram of a multi-level matching process performed by a matching server of a telehealth platform in accordance with the disclosure.

FIG. 3 is a diagram of a first matching pass performed by a matching server of a telehealth platform in accordance with the disclosure.

FIG. 4 is a diagram of a second matching pass performed by a matching server of a telehealth platform in accordance with the disclosure.

FIG. 5 is a diagram of a multi-level matching process performed by a matching server of a telehealth platform in accordance with the disclosure.

FIG. 6 is a state-flow diagram of a reservation process performed by a server of a telehealth platform in accordance with the disclosure.

FIG. 7 is a diagram of a user interface displayed by a management terminal of a telehealth platform in accordance with the disclosure.

FIG. 8 is a diagram of another user interface displayed by a management terminal of a telehealth platform in accordance with the disclosure.

FIG. 9 is a diagram of yet another a user interface displayed by a management terminal of a telehealth platform in accordance with the disclosure.

FIG. 10 is a diagram of a computing device in accordance with the disclosure.

### DETAILED DESCRIPTION

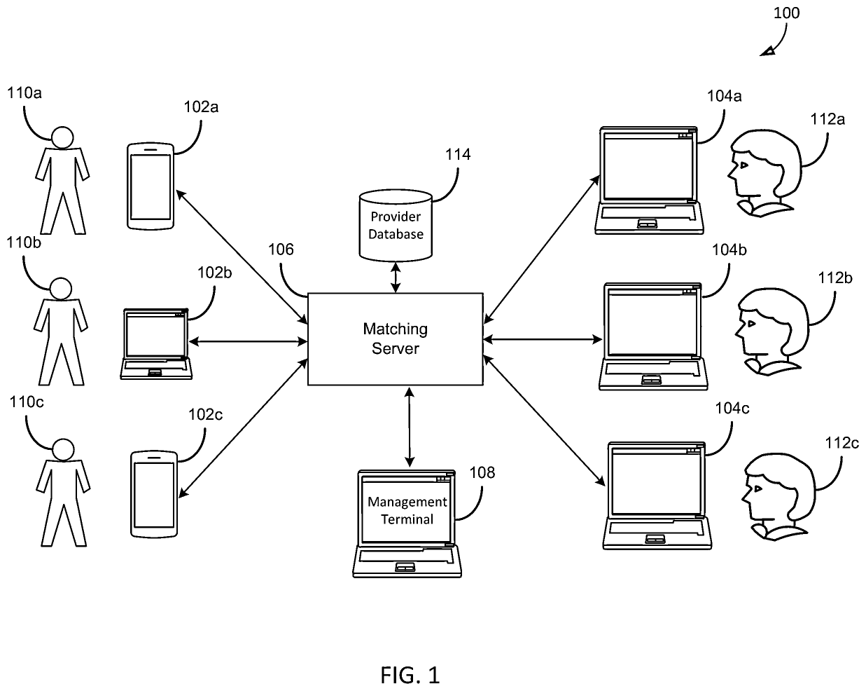

FIG. 1 illustrates an example of a telehealth system 100 in accordance with the disclosure. The system 100 includes a plurality of patient devices 102a-c, a plurality of provider devices 104a-c, at least one matching server 106, and at least one management terminal 108. Each patient device may be associated with a healthcare patient or member 110a-c. Each provider device may be associated with a healthcare provider 112a-c. The matching server 106 may include or otherwise be in communication with a provider database 114. The provider database 114 may store profiles associated with each healthcare provider 112a-c.

The patient devices 102a-c and provider devices 104a-c may take the form smartphones, laptops, desktops, or other personal electronic devices configured to run software that enables the user to interact with the telehealth system 100. The software may take the form of specialized application or a web browser. The patient devices 102a-c, provider devices 104-a-c, matching server 106, management terminal 108, and the provider database 114 may all be coupled to each other via a network, such as the internet.

In general, one or more of the patients 110a-c may user their devices 102a-c to request a consultation with a healthcare provider. The matching server 106 receives the request from the patient device 102 and, using the provider database 114, matches the request to an appropriate healthcare provider 112. The system 100 then establishes a communication session between the patient device 102 and the provider device 104 of the matched healthcare provider 112 to allow the patient and the provider to communicate with each other. The communication session may include any combination of text messaging, audio communication, and/or video communication.

The management terminal 108 may be any computing device configured with a specialized application or web browser that enables a user to interface with the matching server 106. The management terminal 108 provides an interface that allows the user to monitor, configure, and otherwise control the matching server 106. For example, the management terminal 108 may display reports detailing various metrics associated with the telehealth system 100. The management terminal 108 may also allow the user to configure or modify the matching rules executed by the matching server 106, modify provider profiles stored in the provider database 114, and/or perform any other administrative or backend functions associated with the telehealth system 100.

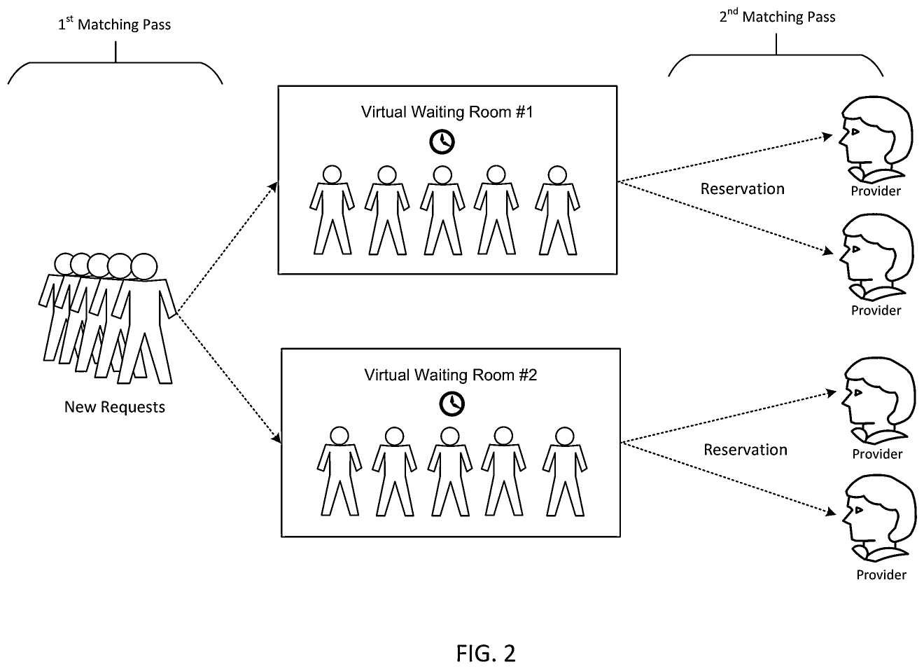

FIG. 2 is a high-level overview of the process performed by the matching server. The process of matching new consultation requests with available providers is broken down into at least two stages or passes. The first pass groups similar consultation requests into virtual waiting rooms. The second pass prioritizes the requests within each waiting room.

Each new or incoming consultation request may include a number of consultation criteria. These criteria may include the location or jurisdiction of the patient (e.g., state, province, country, etc.), the type of consultation requested (e.g., general medical, dermatology, pediatric, mental health, etc.), the patient's insurance information, the modality of the consultation requested by the patient (e.g., text, audio, video, etc.), and/or any other relevant criteria. This information may be supplied directly by the patient when he or she requests the consult. For example, the user may be prompted to complete a form or survey on his or her device upon requesting a consult. Alternatively, the consultation criteria may be derived from other sources, such as a patient profile or electronic medical record stored on the patient device or a remote server. Some consultation criteria, such as location, may be obtained from the patient's device directly.

The matching server examines the consultation criteria of each incoming request and determines whether a virtual waiting room that satisfies the consultation criteria already exists. If so, the incoming request is added to that virtual waiting room. If not, the matching server creates a new virtual waiting room that satisfies the consultation criteria and adds the incoming request to the new virtual waiting room. This process is repeated for all new incoming consultation requests. In addition, the server searches the provider database to identify available providers to assign to the newly created waiting room.

The provider profiles stored in the provider database may include at least the following information, which may be used in determining which providers may be assigned to each waiting room:
A unique provider ID;
Service lines or medical specialties of the provider (e.g., general medicine, dermatology, mental health, etc.);
Licenses or jurisdictions in which the provider is permitted to practice;
Provider network association(s);
Supported modalities (e.g., text messaging, audio, video);
Patient/member age limits (e.g., 18 years and older);
Languages spoken (e.g., English, French, Spanish, etc.);
Pediatrics (whether the provider can service pediatric consults);
Provider affiliation (e.g., employee, medical group contractor, freelance contract, etc.)
On-shift schedules (e.g., days and times when the provider is available to service consults).

Thus, at any given time, the server may maintain a distinct virtual waiting room for each unique combination of consultation criteria found across the pool of requests waiting to be serviced by a provider. Thereafter, the server may sort the requests within each waiting room according to a priority scheme, such as urgency, and reserve the request to providers assigned to that waiting room in accordance with the priority scheme and/or other preference criteria.

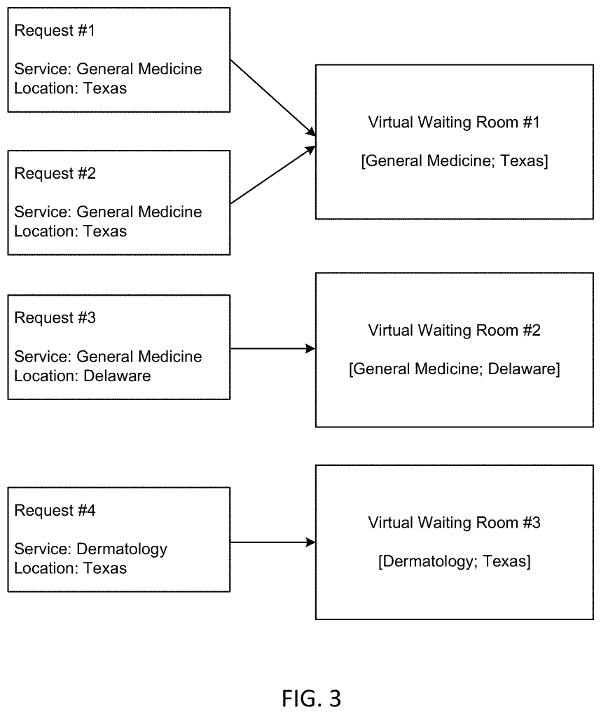

FIG. 3 illustrates an example of the first matching pass performed by the matching server. In this example, the server receives four requests with various consultation criteria in sequential order: Request #1 and Request #2 are for general medicine consults from patients in Texas; Request #3 is for a general medicine consult from a patient in Delaware; and Request #4 is for a dermatology consult from a patient in Texas.

Assume, for this example, that no waiting rooms exist in the server when Request #1 is received. When Request #1 is received by the matching server, the server looks for a waiting room with the following set of consultation criteria: General Medicine and Texas (TX). When the server does not find one (because none previously existed), the server creates Virtual Waiting Room #1 with the corresponding set of criteria and adds Request #1 to Virtual Waiting Room #1.

When Request #2 is received, the server again looks for an existing virtual waiting room for General Medicine in Texas. Assuming that Virtual Waiting Room #1 still exists in the server, the server identifies it as matching the consultation criteria of Request #2 and adds Request #2 to Virtual Waiting Room #1. In general, as long as Virtual Waiting Room #1 exists in the server, every subsequent request for a General Medicine consult in Texas will be added to it (absent some other rule that would override this logic).

When Request #3 is received, the server searches for a virtual waiting room for General Medicine in Delaware. The only other waiting room in the server at this point is Virtual Waiting Room #1, which is also for General Medicine but for Texas, not Delaware. Accordingly, the server creates a new virtual waiting room—Virtual Waiting Room #2—for Request #3 and adds Request #3 to Virtual Waiting Room #2.

Finally, upon the server's receipt of Request #4, the server searches for a virtual waiting room for a dermatological consultation in Texas. The only other waiting room in the server at this point for consultations in Texas is Virtual Waiting Room #1, which is for general medicine consultations, not dermatological consultations. Accordingly, the server creates Virtual Waiting Room #3 for dermatology consultations in Texas and adds Request #4 to Virtual Waiting Room #3.

The above example provides a general description of the logic of the first matching pass performed by the matching server. It is to be understood that exceptions to this logic may be implemented. For example, a system administrator may configure the matching server with an “overflow” rule that causes the server to divert new requests that would otherwise be assigned to a dermatology waiting room to a general medicine waiting room if the backlog of request in the dermatology waiting room exceeds a specified threshold.

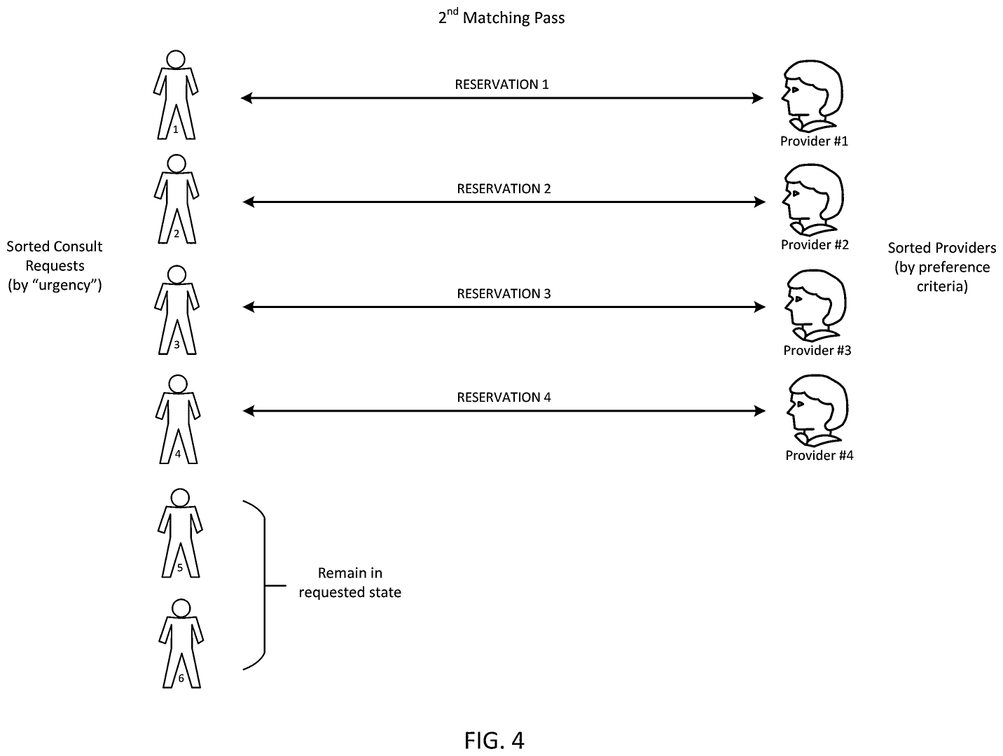

FIG. 4 illustrates the general logic of the second matching pass performed by the matching server, which functions to sort the consult requests in each virtual waiting room according to some priority scheme. The sorting can occur periodically at any appropriate interval, e.g., one second, five seconds, thirty seconds, one minute, five minutes, twenty minutes, etc. The length of the interval may be based an average rate at which new consults requests are received. Alternatively, sorting or re-sorting may be triggered by an event, such as the addition of a new request to the waiting room and/or removal of an existing request from the virtual waiting room.

In one example, the consultations in each virtual waiting room may be sorted according to urgency. That is, each virtual waiting room may be treated as a queue for the next available provider assigned to that waiting room with the highest urgency consult request at the front of the queue and the lowest urgency consult request at the end of the queue. The urgency of a consultation may be evaluated in different ways. In one embodiment, urgency may be calculated based on one or more factors, including age of the request (i.e., time elapsed since the patient submitted the request);
time-to-SLA (Service Level Agreement), i.e., a time remaining until a time by which the request was expected to be fulfilled;
number of available providers assigned to the same waiting room;
spread of the providers assigned to the same waiting room (i.e., how many other requests they may have to service);
likelihood of an SLA breach occurring in the request's waiting room during the foreseeable future.
   The server may assign weights to each of these factors in calculating the urgency of the consult.

The weights applied to each factor of the urgency calculation may be predefined and/or configurable by an administrator. For example, an administrator may be able to select among a number of predefined weighting schemes designed to prioritize different conditions in order to achieve specific healthcare or business goals (or otherwise optimize the service). Moreover, different sorting or priority schemes may be assigned to different virtual waiting rooms (or groups of virtual waiting rooms) at any time. Accordingly, different goals can be achieved in, for example, different geographical regions of the service area or across different service lines (i.e., medical specialties) of the overall customer base. Further, different priority schemes may be assigned to some or all waiting rooms to optimize the service based on seasonal or transient conditions that may influence demand, such as a flu season, “slow season”, pandemic, natural disaster, or other conditions or one-off events. Additionally or alternatively, urgency may be evaluated based on a patient's condition, which may be determined based on responses to a survey or questionnaire or any other method of assessment.

The left side of FIG. 4 shows a number of consult requests in a virtual waiting room. The right side of FIG. 4 illustrates a number of providers that have been assigned to the virtual waiting room. The consult requests have been sorted according to urgency, as discussed above. The available providers assigned to this waiting room are also sorted according to certain preference criteria, discussed in more detail below. Once the consults requests and the assigned providers have been sorted, the matching server will, starting with the highest priority consult request in the virtual waiting room, reserve that consult request to the highest priority provider assigned to the virtual waiting room. This process may repeat until all available providers assigned to the virtual waiting room have been reserved for a consultation request in that virtual waiting room. Thus, after sorting the requests and the providers shown in FIG. 4, Request #1 is reserved for Provider #1 as Reservation #1, Request #2 is reserved for Provider #2 as Reservation #2, and so on, through Reservation #4. Requests 5 and 6 remain in an unreserved state until another provider assigned to the room is available to accept a new reservation.

The preference criteria used by the matching server to sort the providers assigned to a waiting room may include any number of attributes, including the following, which may be stored in a profile associated with each provider in the provider database:
Routing rule(s)—rules that may be used to define supersets of providers that can service a particular consult request; these supersets may be specific medical groups or other associations of providers;
Languages spoken by the provider;
Pediatrics—i.e., whether the provider will see patients under a certain age (e.g., 18);
Provider affiliation—the legal relationship between the provider and telehealth service provider (for example, the service provider may prioritize providers who are employees over those who are contractors, and those who are contracted as part of certain medical groups over freelance contractors);
On-call status—i.e., whether the provider is on-shift at the current time (e.g., providers who are on-shift may be prioritized over those who are off-shift);
Longest provider wait time—this can be used to prioritize providers based on wait times associated with each provider (e.g., providers with shorter wait times may be prioritized over those with longer wait times).

In one embodiment, the providers may be sorted using the above criteria in the order in which they are listed—i.e., the providers are first sorted according to routing rule, then language, then pediatrics, etc. However, the above criteria, or any subset of those criteria, and/or any other criteria may be used alone or in any order to sort the providers assigned to a particular waiting room.

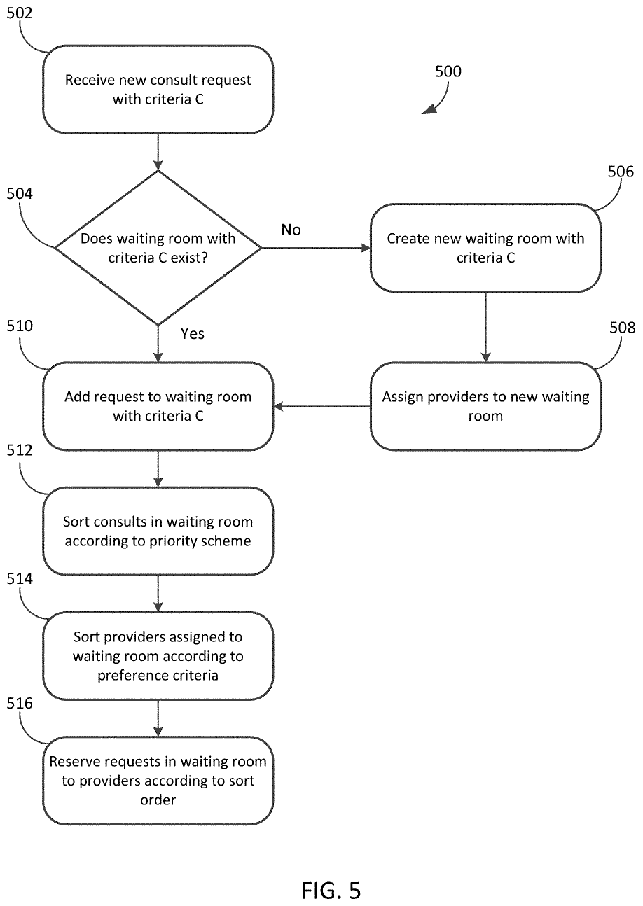

FIG. 5 is a flow chart illustrating a matching process 500 performed by the matching server including the first and second pass matching described above. At step 502, a new consultation request is received at the server and includes a set of consultation criteria C. At step 504, the server determines whether a virtual waiting room for the set of criteria C already exists in the server. If not, the server proceeds to step 506, where it creates a new virtual waiting room having the criteria C.

At step 508, the server searches the provider database to determine which available providers can service consultation requests having the set of criteria C and assigns those providers to the newly created waiting room. The server then proceeds to step 510 where it adds the new request to the newly created waiting room with criteria C. Alternatively, if the server had determined at step 504 that a waiting room having the criteria C already existed in the server, the server would have proceeded directly from step 504 to step 510, where the new request is added to the virtual waiting room having criteria C.

At step 512, the server sorts the requests in the waiting room according to a priority scheme. As discussed above, the priority scheme may be an urgency score based on a weighted combination of various factors.

At step 514, the server sorts the providers assigned to the waiting room according to their preference criteria, as discussed above with respect to FIG. 4. For example, the providers assigned to the waiting room may be ranked or sorted according to their routing rule(s), languages, and longest wait time, and other criteria.

At step 516, the server sequentially reserves the highest priority unreserved consult in the waiting room to the most preferred unreserved provider assigned to the waiting room repeatedly until all of the providers assigned to the waiting room have been reserved to a consult request. As discussed in more detail below, when a provider completes a reserved consultation, the server may change the status of the provider to unreserved and then reserve the next highest priority unreserved consultation request in the waiting room to that provider.

This process 500 is an example intended to illustrate the basic flow of a two-stage matching process. It should not be construed as a rigid or complete software process diagram. For example, the sorting of consults according at step 512 need not necessarily occur before the sorting of providers at step 514. Indeed, these processes may occur in any order or in parallel in a preferred implementation.

The server may be configured to delete a waiting room once all the requests in that waiting room have been serviced. In this way, the server need only maintain a waiting room object in memory so long as there is an active request within it waiting to be serviced. In an alternative embodiment, although the above-described process shows the server creating new waiting rooms when requests with new combinations of consult criteria are received, the server could also be configured to create empty waiting rooms for every possible combination of consultation request criteria on startup and simply add new consults to the already created waiting rooms when they are received. Each of the virtual waiting room may be implemented as a table in any suitable database software.

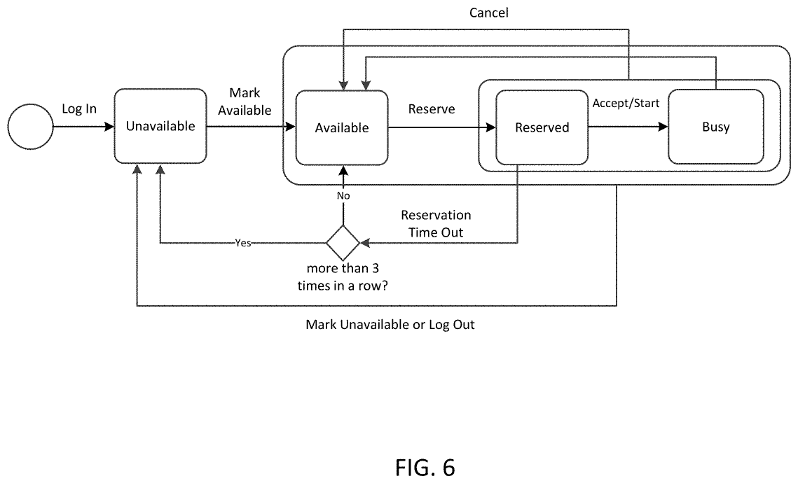

FIG. 6 is a state diagram illustrating a logic that may be implemented by the server for determining a provider's status or “availability.” In general, a provider may be treated as unavailable unless they have logged into the service and selected an option in the provider's user interface to mark themselves as “available.” Once this occurs, the server may assign the provider to one or more waiting rooms that the provider can service.

As described above, once assigned to a waiting room, the server may reserve a consultation request in the waiting room to the provider. At that point, the provider has a predefined period of time (e.g., 90 seconds) during which to accept or otherwise start the consult reserved to them. If they do, they are marked busy. If the provider does not accept or start the reserved consult within the predefined time period, the server may return the consult request to an unreserved state and return the provider to the available state. If the provider fails to accept more than a predefined number of consults (e.g., 3) in a row, the server may mark the provider as unavailable and remove any waiting room assignments.

Once the provider starts a consult reserved to them, he or she is marked as busy by the server. When the consult ends, the provider is once again marked as available and will be reserved a new consultation request by the server according to the process described above.

In one embodiment, when a consult request is reserved, the server may communicate with a conferencing platform to reserve or otherwise create a conferencing (such as videoconferencing) room or resource in which the consultation will take place. The server may then transmit a message to the patient either through the patient app or a text message that includes a URL or other link that will direct the patient device software to the conferencing room where the consultation will occur. When the provider selects to start a reserved consultation within the provider software, a similar link or URL may direct the provider software to the reserved conferencing room to initiate the conference between the patient and the provider.

As discussed above, providers may be associated with routing rules that can be used to sort or otherwise prioritize providers assigned to any particular waiting room. A routing rule may associate a group of providers with a priority level for a particular region, such as a state, country, territory, or province. The group of providers associated with each routing rule may be affiliated with each other through a business or healthcare organization. Thus, where multiple groups of providers can service a particular waiting room, routing rules allow the telehealth service provider to give a first group of providers priority over a second group of providers when reserving consult requests to the superset of providers. For example, if providers associated with provider network ABC and providers associated with provider network XYZ are assigned to a waiting room in Alaska, and the routing rule for provider network ABC in Alaska is higher than that of the routing rule for network XYZ in Alaska, the server will reserve consultation requests to providers from network ABC before reserving consult requests to providers from network XYZ. At the same time, another routing rule may give providers from network XYZ a higher priority than providers from network ABC in a different location, such as California. This allows the telehealth service provider to more effectively manage relationships with provider groups and/or payers, such as Medicare or Medicaid.

Routing rules can also be used to manage transient conditions such as demand surges in a particular area. For example, if the server detects that wait times for consults in a particular waiting room have exceeded a predefined threshold (e.g., 60 minutes), routing rules can be used to force new requests that would otherwise be placed in the same waiting room into a different waiting room with shorter wait times to help service the consult requests more quickly.

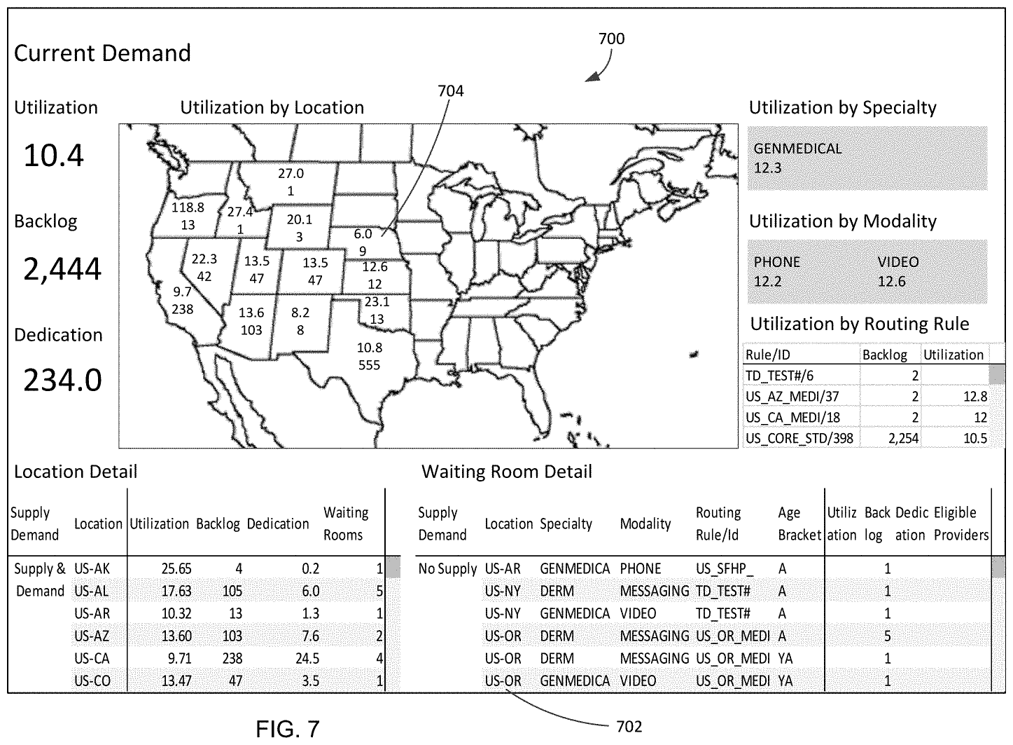

FIG. 7 shows an example of a command center user interface 700 that may be displayed by a management terminal or other device. The interface may be configured to display a variety of information received from the matching server, such as utilization, backlog, and dedication. These metrics may be broken down or visualized in various ways, including by geographic location, specialty, modality, and/or routing rule. The interface 700 may also display provide details about locations and waiting rooms. In one embodiment, the waiting room detail may include a scrollable list of all waiting rooms 702 currently maintained by the matching server. Each row in the list may be an interactive element that can be selected by the user to open a details page for the selected waiting room that may include further details about the waiting room and allow the user to make changes to the routing rule, priority scheme, or other configurable attributes of the waiting room. The interface 700 may also allow the user to search, filter, or otherwise select any number of waiting rooms to make configuration changes to at once.

Interface 700 may also include an interactive graphical display window 704 of a service area that displays aggregate usage metrics relating to any waiting rooms or jurisdictions within the service area. As shown in FIG. 7, display window 704 may include aggregate utilization metrics in, e.g., some or all U.S. states and may allow the user to click within any of these jurisdictions to open a new window that shows further details and configuration options related to the selected jurisdiction.

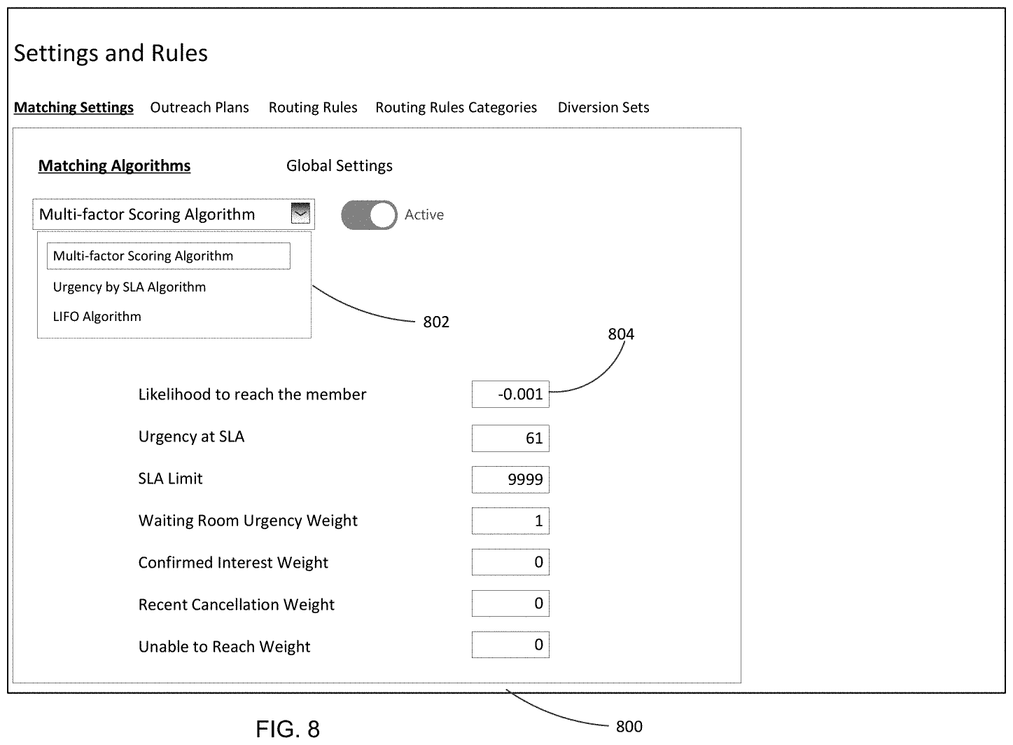

FIG. 8 is an example of a user interface 800 that may displayed to configure the priority scheme or matching algorithm associated with of one or more virtual waiting rooms. This interface may be displayed when, for example, the user clicks one or more waiting rooms displayed in the list 702 in FIG. 7. The interface 800 may include a drop-down menu 802 or other graphical element that allows the user to select among several matching algorithms to assign to the selected one or more waiting rooms. If the selected algorithm includes user-configurable settings, the interface 800 may include one or more input fields 804 to allow the user to enter weights and/or other information to configure the algorithm. In this way, the interface 800 allows the user to quickly and easily toggle between and/or modify priority schemes in order to optimize the behavior of the system to meet changing demands. Although certain weighting factors are illustrated in FIG. 8, any number of different factors could be used. The factors illustrated in FIG. 8 are exemplary only and defined as follows:
Likelihood to reach the member: This weighting may be used to determine the rate at which the likelihood of reaching the member decreases as the consult ages.
Urgency at SLA: This value may be used to set the urgency of a consult at the moment the SLA expires.
SLA Limit: This value may be used to limit SLAs to the entered duration in minutes.
Waiting Room Urgency Weight: This weighting may be used to determine the degree to which a waiting room's most pessimistic wait time factors into the urgency of its consults.
Confirmed Interest Weight: This weighting may be used to determine the matching preference given to members who confirm their interest to conduct the consultation. This confirmation may be obtained by members activating a link transmitted to the member via text message.
Recent Cancellation Weight: This weighting may be used to determine the matching preference given to members whose consults were cancelled for any reason during a preceding 24 hour period.
Unable to Reach Weight: This weighting may be used to determine the decrease in urgency for consults where the provider was unable to reach the member.

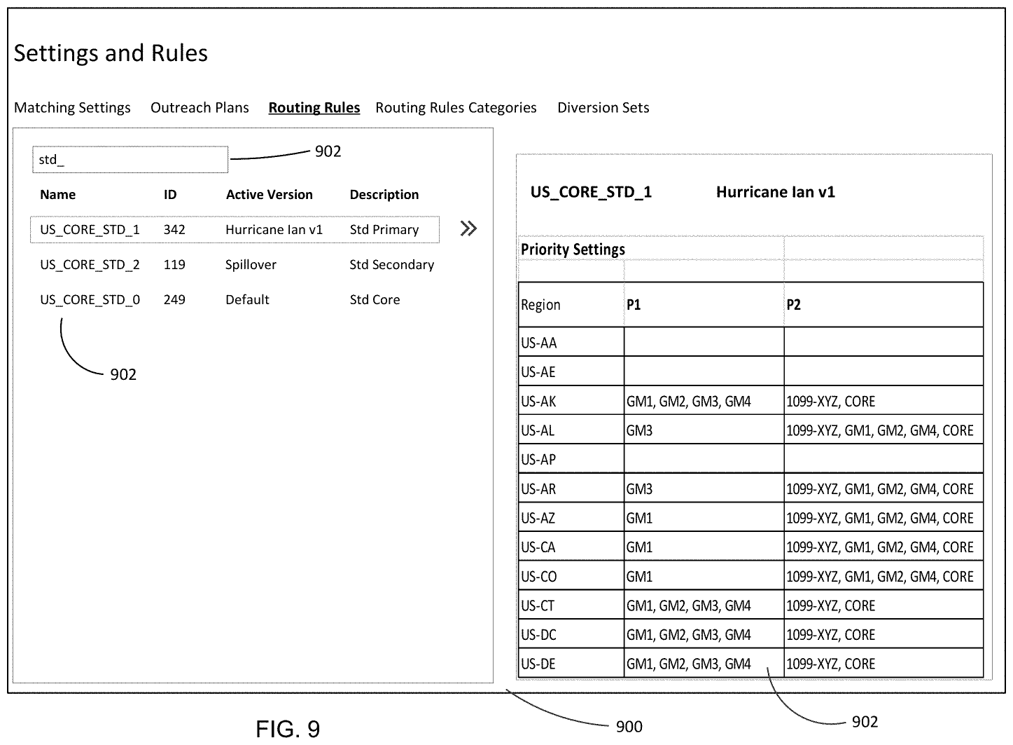

FIG. 9 is an example of a user interface 900 that may be displayed by the management terminal to allow a user to define or reconfigure routing rules. The interface 900 may include a search box 902 that allows the user to filter and/or search for a particular routing rule by name. When a rule is selected from the list of rules 902, the interface may display a window 902 that allows the user to view and/or edit the priority level and/or locations associated with various provider groups. For example, from window 902, the user could edit the routing rule such that provider group “GM1” is moved from priority level 2 (“P2”) to priority level 1 (“P1”) in the state of Alabama.

As noted above, one factor that may be considered in sorting consults in a waiting room by urgency is the “spread” of the providers assigned to the same waiting room. The spread value of any provider may be calculated as the sum, across all waiting rooms to which the provider is assigned, of the number of consult requests in each waiting room divided by the number of providers assigned to each waiting room, or

$${Spread}_{P} = {\sum }_{WR} \frac{Number of requests in waiting room}{Number of providers assigned to waiting room}$$

where P is a provider and WR is the set of waiting rooms to which provider P is currently assigned.

The provider spread metric can be used to more efficiently matching consult requests with the available providers. For example, assume a waiting room for consults in Texas has eight pending consults requests and another waiting room for Delaware has one pending consult in it. Now assume there are four available providers: three with Texas licenses assigned to the Texas waiting room only and a fourth available provider with both Texas and Delaware licenses assigned to both waiting rooms. The provider spread of each of the three providers with only Texas licenses would be 2 (i.e., eight consults in the Texas waiting room divided by four providers assigned to the Texas waiting room). The provider spread of the provider with both Texas and Delaware licenses, however, is 3: eight consults in the Texas waiting room divided by four providers assigned to that waiting room—i.e., two—plus one consult in the Delaware waiting room divided by one provider assigned to that waiting room. Thus, the provider spread metric reveals that the dual-license provider is spread more thinly (i.e., has more possible consults to service) than the Texas-only providers. The difference in provider spread can be used by the server to first give consults in the Texas waiting room to the Texas-only providers, thereby allowing the dual-license provider more opportunity to service consults in the Delaware waiting room. This is important in cases where a multi-state provider is assigned to multiple waiting rooms with significantly different demand levels. If provider spread were not considered, the multi-state provider may end up only servicing requests from the higher-demand waiting room, which can cause unnecessarily long wait times for requests in the lower-demand waiting room.

In order to use the provider spread metric to sort consults in a waiting room by urgency, the server may be configured to use the provider spread metric to estimate a wait time for each consult in the waiting room. To do this, the server may first calculate a “provider dedication” value for each waiting room. The provider dedication value of a given waiting room may be calculated as the sum, across all providers assigned to the waiting room, of the product of each provider's spread value and the number of requests in the waiting room divided by the number of providers assigned to the waiting room, or

$$Provider {Dedication}_{WR} = {\sum }_{{P}_{WR}} S p r e a {d}_{P} * \frac{Number of consultants in WR}{Number of providers assigned to WR}$$

where P_{WR} is the set of providers assigned to a waiting room WR and Spread_{P} is the spread of a particular provider P assigned to the waiting room WR.

Returning to the example discussed above with respect to provider spread, the Texas waiting room has a provider dedication value of 3.67

$$( i . e . , ( \frac{2}{2} ) + ( \frac{2}{2} ) + ( \frac{2}{2} ) + ( \frac{2}{3} ) )$$

while the Delaware waiting room has a provider dedication value of 0.33 (i.e., (i)). Using these provider dedication values, the server may estimate a maximum wait time for each consult request as follows:

$${Wait}_{R} = D * Ceiling ( \frac{Index}{{Dedication}_{p}} - 1 )$$

where Wait_{R} is the estimated maximum wait time for a request in a waiting room, D is an expected visit duration (e.g., 10 minutes), “Index” is an integer representing a number of requests ahead of the current consult request in the waiting room, and “ceiling” is a function that rounds up to the nearest integer. Thus, all other things being equal, the greater the provider dedication value in a given waiting room, the lower the maximum estimated wait time for a consult request in that waiting room.

Once the server has calculated maximum estimated wait times for each consult in a waiting room, the server can calculate the relative urgency of each consult in the waiting room by subtracting the time-to-SLA from the maximum estimated wait time for each consult. The SLA or “service level agreement” for a request may depend on contractual obligations between the patient or member, their benefits provider, and/or the telehealth service provider. For example, some patients may have a benefits package that guarantees a maximum wait time for a consult of fifteen minutes or less, while other members may have a benefits package that guarantees a maximum wait time of thirty minutes or less. Thus, the time-to-SLA for each consult request may vary depending not only on the age of the request but also the service level of each request. In any case, once the relative urgency of each request in the waiting room is known, the server can sort the requests in the waiting room according to urgency.

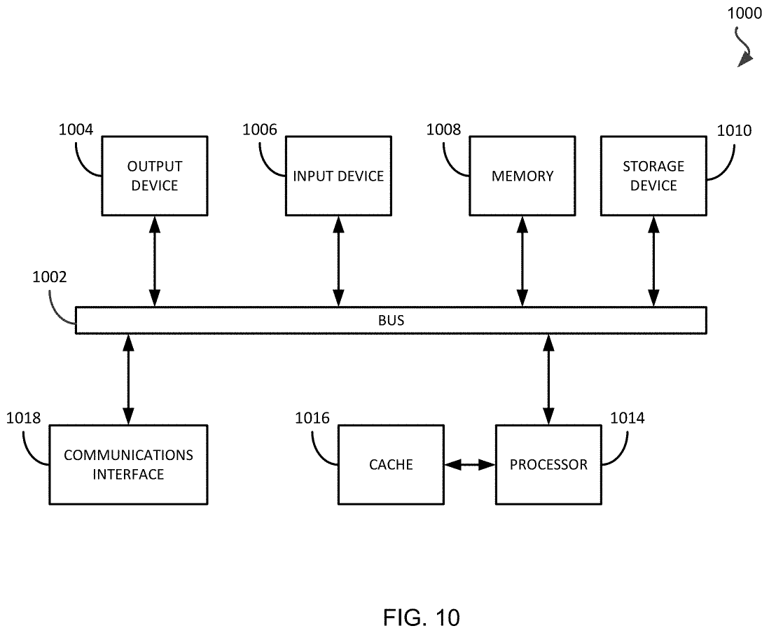

FIG. 10 depicts an example computer system 1000 that may implement various systems and methods discussed herein. The computer system 1000 includes one or more computing components in communication via a bus 1002. In one implementation, the computer system 1000 includes one or more processors 1014. Each processor 1014 may include one or more internal levels of cache 1016, as well as bus controller or bus interface unit to direct interaction with a bus 1002.

A memory 1008 may include one or more memory cards and control circuits (not depicted), or other forms of removable memory, and may store various software applications including computer executable instructions, that when run on the processor 1014, implement the methods and systems set out herein. Other forms of memory, such as a mass storage device 1010, may also be included and accessible, by the processor (or processors) 1014 via the bus 1002.

The computer system 1000 may further include a communications interface 1018 by way of which the computer system 1000 can connect to networks and receive data useful in executing the methods and system set out herein as well as transmitting information to other devices. The computer system 1000 may include an output device 1004, such as graphics card or other display interface by which information can be displayed on a computer monitor. The computer system 1000 can also include an input device 1006 by which information is input. Input device 1006 can be a mouse, keyboard, touchscreen, scanner, and/or other input devices as will be apparent to a person of ordinary skill in the art.

The system set forth in FIG. 10 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magnetooptical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in any claims that follow.

What is claimed is:

1. A method for establishing electronic communication sessions between patients and healthcare providers, comprising:
receiving, at a server via a network, a plurality of consultation requests from a plurality of patient devices, each request including patient-supplied consultation criteria;
identifying, using a processor, each unique combination of consultation criteria among the plurality of consultation requests received at the server;
generating, within the server, a virtual waiting room for each unique combination of consultation criteria;
assigning, within the server, each consultation request of the plurality of consultation requests to the virtual waiting room with matching consultation criteria;
comparing, using the processor, the unique combination of consultation criteria of each virtual waiting room with a profile for each provider stored in a provider database, each provider having a respective provider device;
assigning each provider whose profile satisfies a unique combination of consultation criteria to a corresponding virtual waiting room within the server;
sorting, using the processor, the consultation requests for each virtual waiting room according to a consultation priority scheme; and
establishing, via the network, an electronic communication session between a patient device associated having a highest priority consultation request within each virtual waiting room and a provider device associated with a first available provider assigned to the corresponding virtual waiting room.

2. The method of claim 1, wherein a provider is an available provider until an application running on the provider device is stopped or the provider fails to accept a predetermined number of video calls within a predetermined period of time.

3. The method of claim 1, wherein the electronic communication session is initiated without requiring the provider to request a consultation.

4. The method of claim 1, wherein the consultation priority scheme is based on one or more of the following: severity; wait time; language preference; clinical specialty preference; provider licensure; provider loyalty status.

5. The method of claim 4, associating a first consultation priority scheme with a first virtual waiting room and a second consultation priority scheme with a second virtual waiting room, wherein the first and second consultation priority schemes are different.

6. The method of claim 1, further comprising:
sorting the providers assigned to a virtual waiting room in accordance with a provider priority scheme; and
establishing an electronic communication session between a patient device having the highest priority consultation request within the virtual waiting room and a provider device associated with a highest priority provider assigned to the virtual waiting room.

7. The method of claim 1, further comprising displaying, at a management terminal in communication with a matching terminal, a graphical user interface that provides an alert when a condition is met, wherein the condition is a function of one or more of the following:
a number of providers assigned to a virtual waiting room;
a number of consultation requests assigned to a virtual waiting room;
a rate of new consultation requests added to a virtual waiting room; and
a rate of consultations in the virtual waiting room being completed.

8. The method of claim 7, further comprising displaying a list of potential providers whose profiles satisfy the unique consultation criteria associated with the virtual waiting room.

9. A system for establishing electronic communication sessions between patients and healthcare providers, the system comprising:
a matching server in communication with a plurality of patient devices and a plurality of provider devices, the matching server configured to perform the following:
receive, via a network, a plurality of consultation requests from a plurality of patient devices, each request including patient-supplied consultation criteria;
identify, using a processor, each unique combination of consultation criteria among the plurality of consultation requests;
generate a virtual waiting room for each unique combination of consultation criteria;
assign each consultation request of the plurality of consultation requests to the virtual waiting room with matching consultation criteria;
compare, using the processor, the unique combination of consultation criteria of each virtual waiting room with a profile for each provider stored in a provider database, each provider having a respective provider device;
assign each provider whose profile satisfies a unique combination of consultation criteria to a corresponding virtual waiting room;
sort, using the processor, the consultation requests for each virtual waiting room according to a consultation priority scheme; and
establish, via the network, an electronic communication session between a patient device associated having a highest priority consultation request within each virtual waiting room and a provider device associated with a first available provider assigned to the corresponding virtual waiting room.

10. The system of claim 9, wherein a provider is an available provider until an application running on the provider device is stopped or the provider fails to accept a predetermined number of video calls within a predetermined period of time.

11. The system of claim 9, wherein the electronic communication session is initiated without requiring the provider to request a consultation.

12. The system of claim 9, wherein the consultation priority scheme is based on one or more of the following: severity; wait time; language preference; clinical specialty preference; provider licensure; provider loyalty status.

13. The system of claim 12, including a first consultation priority scheme associated with a first virtual waiting room and a second consultation priority scheme associated with a second virtual waiting room, wherein the first and second consultation priority schemes are different.

14. The system of claim 9, wherein the matching server is configured to sort the providers assigned to a waiting room in accordance with a provider priority scheme and establish a communication session between a patient device associated with the highest priority consultation request within the virtual waiting room and a provider device associated with the highest priority provider assigned to the virtual waiting room.

15. The system of claim 9, further comprising a management terminal in communication with a matching terminal, the management terminal is configured to display a graphical user interface that provides an alert when a condition is met, wherein the condition is a function of one or more of the following:
a number of providers assigned to a virtual waiting;
a number of consultation requests assigned to a virtual waiting room;
a rate of new consultation requests added to a virtual waiting room; and
a rate of consultations in the waiting room being completed.

16. The system of claim 15, wherein the graphical user interface displays a list of potential providers whose profiles satisfy the unique consultation criteria associated with the waiting room.

17. A system for establishing electronic communication sessions between patients and healthcare providers, the system comprising:
a management terminal in communication with a matching server in communication with a plurality of patient devices and a plurality of provider devices, the matching server configured to perform the following:
receive, via a network, a plurality of consultation requests from a plurality of patient devices, each request including patient-supplied consultation criteria;
identify, using a processor, each unique combination of consultation criteria among the plurality of consultation requests;
generate a virtual waiting room for each unique combination of consultation criteria;
assign each consultation request of the plurality of consultation requests to the virtual waiting room with matching consultation criteria;
compare, using the processor, the unique combination of consultation criteria of each virtual waiting room with a profile for each provider stored in a provider database, each provider having a respective provider device;
assign each provider whose profile satisfies a unique combination of consultation criteria to a corresponding virtual waiting room;
sort, using the processor, the consultation requests for each virtual waiting room according to a consultation priority scheme; and
establish, via the network, an electronic communication session between a patient device associated having a highest priority consultation request within each virtual waiting room and a provider device associated with a first available provider assigned to the corresponding virtual waiting room;
wherein the management terminal is configured to display a graphical user interface that provides an alert when a condition is met.

18. The system of claim 17, wherein the condition is a function of one or more of the following:
a number of providers assigned to a virtual waiting;
a number of consultation requests assigned to a virtual waiting room; and
a rate of new consultation requests added to a virtual waiting room.

19. The system of claim 18, wherein the graphical user interface displays a list of potential providers whose profiles satisfy the unique consultation criteria associated with the virtual waiting room.

20. The system of claim 17, wherein a provider is an available provider until an application running on the provider device is stopped or the provider fails to accept a predetermined number of video calls within a predetermined period of time.

21. The system of claim 17, wherein the electronic communication session is initiated without requiring the provider to request a consultation.

22. The system of claim 17, wherein the consultation priority scheme is based on one or more of the following: severity; wait time; language preference; clinical specialty preference; provider licensure; provider loyalty status.

23. The system of claim 22, including a first consultation priority scheme associated with a first virtual waiting room and a second consultation priority scheme associated with a second virtual waiting room, wherein the first and second consultation priority schemes are different.

24. The system of claim 17, wherein the matching server is configured to sort the providers assigned to a waiting room in accordance with a provider priority scheme and establish a communication session between a patient device associated with the highest priority consultation request within the virtual waiting room and a provider device associated with the highest priority provider assigned to the virtual waiting room.
